(19) United States
(12) Patent Application Publication
(10) Pub. No.: US 2015/0209597 A1
(43) Pub. Date: Jul. 30, 2015

(54) PHOTOTHERAPY EYEGLASS DEVICE

(71) Applicants: MICHAEL E. HAARLANDER, VERO BEACH, FL (US); THOMAS M. HAARLANDER, VERO BEACH, FL (US)

(72) Inventors: MICHAEL E. HAARLANDER, VERO BEACH, FL (US); THOMAS M. HAARLANDER, VERO BEACH, FL (US)

(21) Appl. No.: 14/560,601

(22) Filed: Dec. 4, 2014

Related U.S. Application Data
(60) Provisional application No. 61/911,644, filed on Dec. 4, 2013.

(51) Int. Cl. A61N 5/06; A61H 23/00

(52) U.S. Cl. CPC A61N 5/0618; A61N 5/0603; A61H 23/00; A61N 2005/0605; A61N 2005/0626; A61N 2005/0648

(57) ABSTRACT

A phototherapy device, such as goggles, eye-glasses, or an eye mask, for applying light from a plurality of semiconductor light and/or vibrational sources, such as, but not limited to, light-emitting diodes (LEDs) or laser diodes, fiber optics, full spectrum light sources, piezo transducers, vibrational transducers, or a combination thereof, that provide therapeutic and rejuvenating effects to human tissue, to the eyes, ears, face, and head area. The light or vibrational sources may be arrayed on a detachable, interior-facing face plate attached to the device or a head-strap. Various face plates contain various configurations or patterns of light sources, in a variety of colors and intensities. The face plates also may vary in size and shape. One or two ear buds or earphones may be provided with a combination of speakers and light or vibrational sources.

This application claims benefit of and priority to U.S. Provisional Application No. 61/911,644, filed Dec. 4, 2013, by Michael E. Haarlander, et al., and is entitled to that filing date for priority. The specification, figures and complete disclosure of U.S. Provisional Application No. 61/911,644 are incorporated herein by specific reference for all purposes.

### FIELD OF INVENTION

This invention relates to an eyeglass device and related methods for phototherapy or light therapy applications to the eyes and ears of the body.

### BACKGROUND OF INVENTION

Physicists have recognized light to be a pure form of energy that is a part of the electromagnetic radiation spectrum. Within this spectrum, the various colors of light each represent unique wavelengths and frequencies that produce therapeutic effects when absorbed through the skin of the body. Near infrared wavelengths are longer than visible light wavelengths, and have been found to penetrate to a deeper level of muscle, bones and joints. Visible red light tends to stimulate growth at the cellular level whereas blue light has more of a soothing affect.

Ninety-eight per cent of sunlight enters through the eyes, and 2% through the skin. The 2% on the skin is important because that is the only way your body can develop vitamin D which is essential for calcium production to make strong bones and strong teeth. Light entering the body is best delivered through the eyes.

Further scientific research has found that the application of low-level light therapy on the body produces a photochemical reaction in the cell. During this process, a photon of light is absorbed into the treated cell to increase and stimulate stored energy. As a result, this stored energy transforms into chemical energy that can be used to regulate cellular activity; including enhanced ATP synthesis, protein synthesis, cellular proliferation, and growth factor secretion.

Low-level light therapy increases energy; reduces pain and inflammation; and reduces stress, as documented in the following reports, which are incorporated herein by specific reference for all purposes (excerpts and abstracts are of which are attached as an appendix to U.S. Provisional No. 61/911,644, filed Dec. 4, 2013, and incorporated herein by specific reference for all purposes): Geerdink, et al., “Blue Light in the Morning Phase Advances the Rhythym of Melatonin and Reduces Sleepiness at Waking Up.”

Jurvelin, et al., “The Effect of Transcranial Bright Light Treatment via Ear Canals on Human Psychophysiology—A Randomized Controlled Study.”

Tulppo, et al., “Effects of Bright Light Treatment on Psychomotor Speed in Top Level Athletes: Randomized, Double-blind, Placebo Controlled Study.”

Daneault, et al., “Pupil Light Reflex in Response to Monochromatic Light Stimuli in Younger and Older Subjects.”

Figueiro, et al., “Exposure to Daylight As Well As To Blue and Red Lights at Night Interact to Affect Nocturnal Performance, Subjective Sleepiness and Biomarker Production.”

Mustafina, et al., “Bright Light Therapy for Weight Loss,” Soc. Light Treatment Biol. Rhythms 19:7 (2007).

Kunz, et al., “Differential Effects of Short-Term Light Exposure on Melatonin Excretion,” Soc. Light Treatment Biol. Rhythms 19:20 (2007).

Lieverse, et al., “Bright Light Treatment in Elderly Patients With Nonseasonal Major Depressive Disorder: A Randomized Placebo-Controlled Trial,” Arch. Gen. Psych. 68(1): 61-70 (2001).

Vanderwalle, et al., “Abnormal Hypothalamic Response to Light in Seasonal Affective Disorder,” Biol. Psych. 70(10): 954-61 (November 2011).

Karu, et al., “Cellular Effects of Low Power Laser Therapy Can be Mediated by Nitric Oxide,” Lasers Surg. Med. 36(4): 307-14 (April 2005).

Nawashiro, et al., “Focal increase in cerebral blood flow after treatment with near-infrared light to the forehead in a patient in a persistent vegetative state,” Photomed. Laser Surg. 30(4): 231-33 (April 2012).

Naeser, et al., “Improved cognitive function after transcranial, light-emitting diode treatments in chronic, traumatic brain injury: two case reports,” Photomed. Laser Surg. 29(5): 351-58 (May 2011).

### DESCRIPTION OF THE DRAWINGS

FIG. 1 shows a rear view of a phototherapy eyeglass device in accordance with an exemplary embodiment of the present invention.

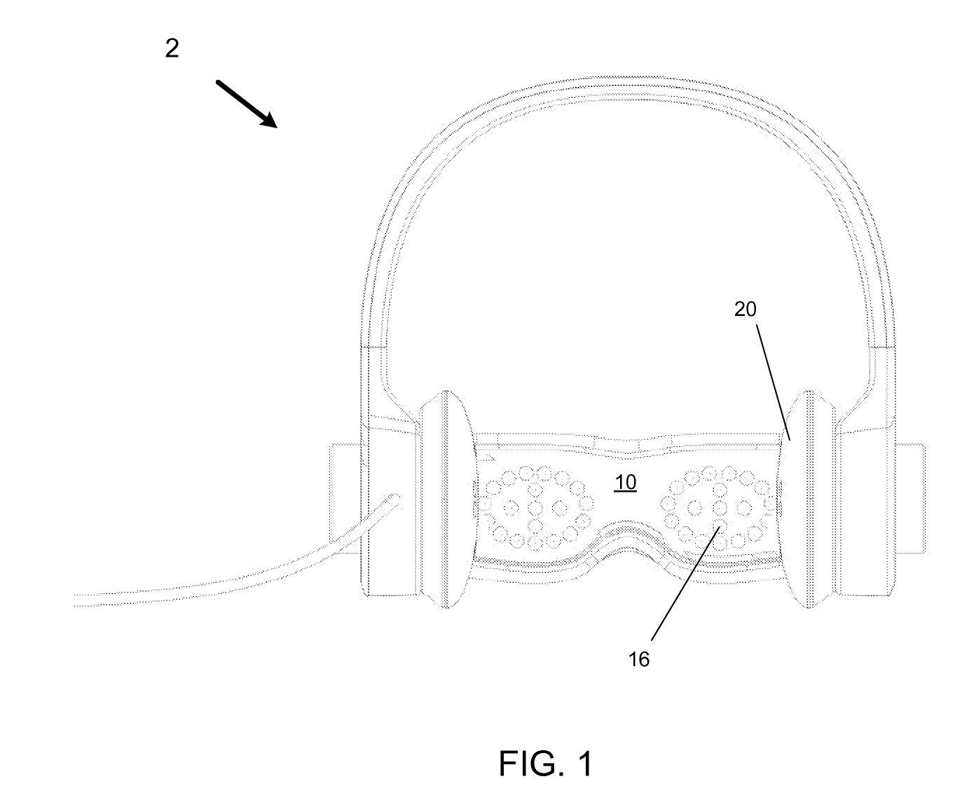

FIG. 2 shows a front view of the device of FIG. 1.

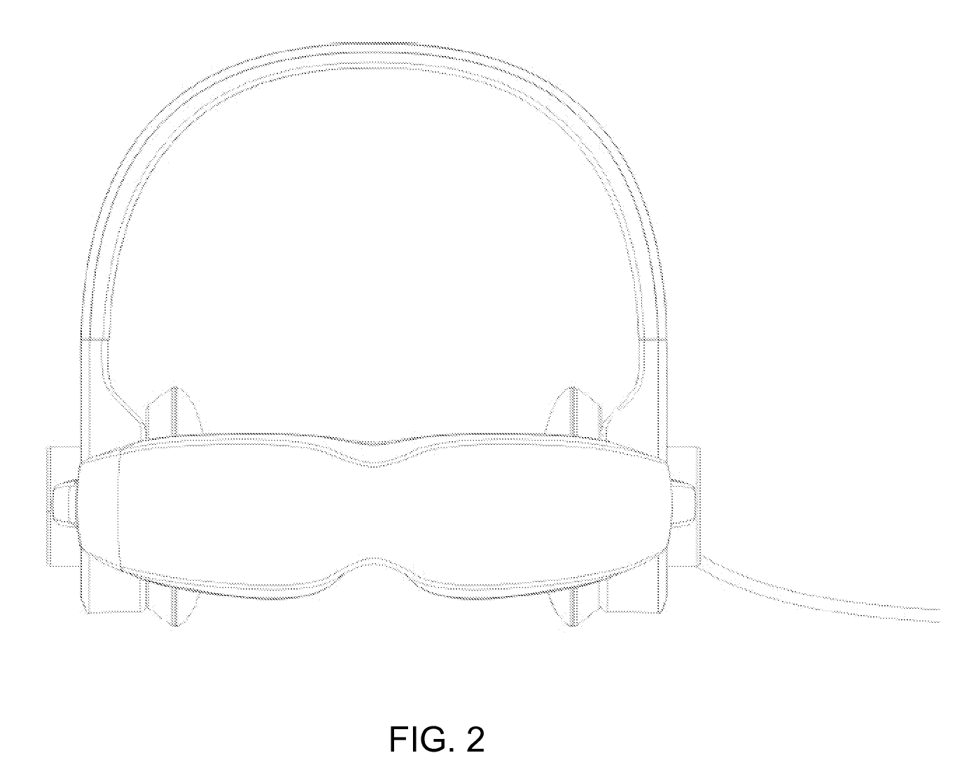

FIG. 3 shows a left side view of the device of FIG. 1.

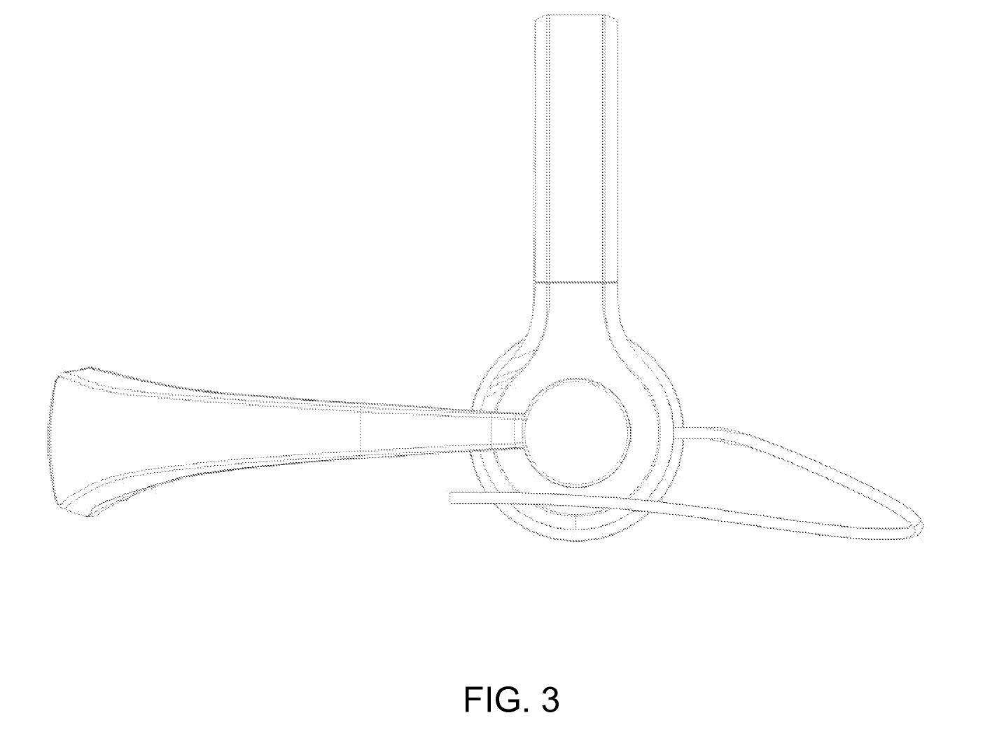

FIG. 4 shows a rear perspective view of the device of FIG. 1.

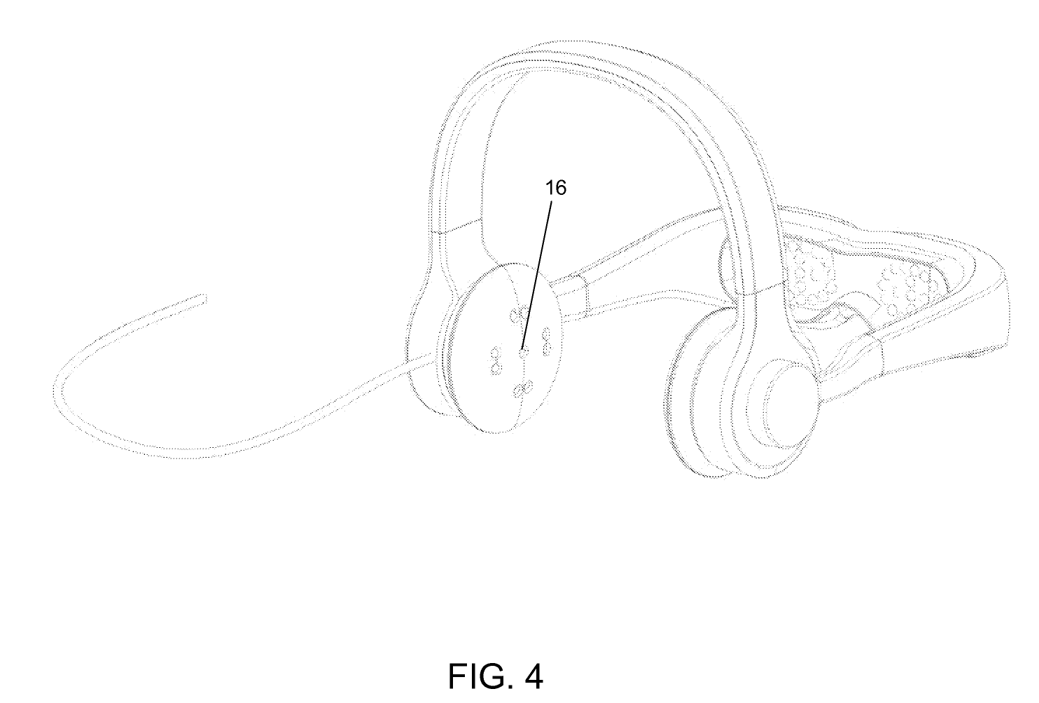

FIG. 5 shows a front perspective view of the device of FIG. 1.

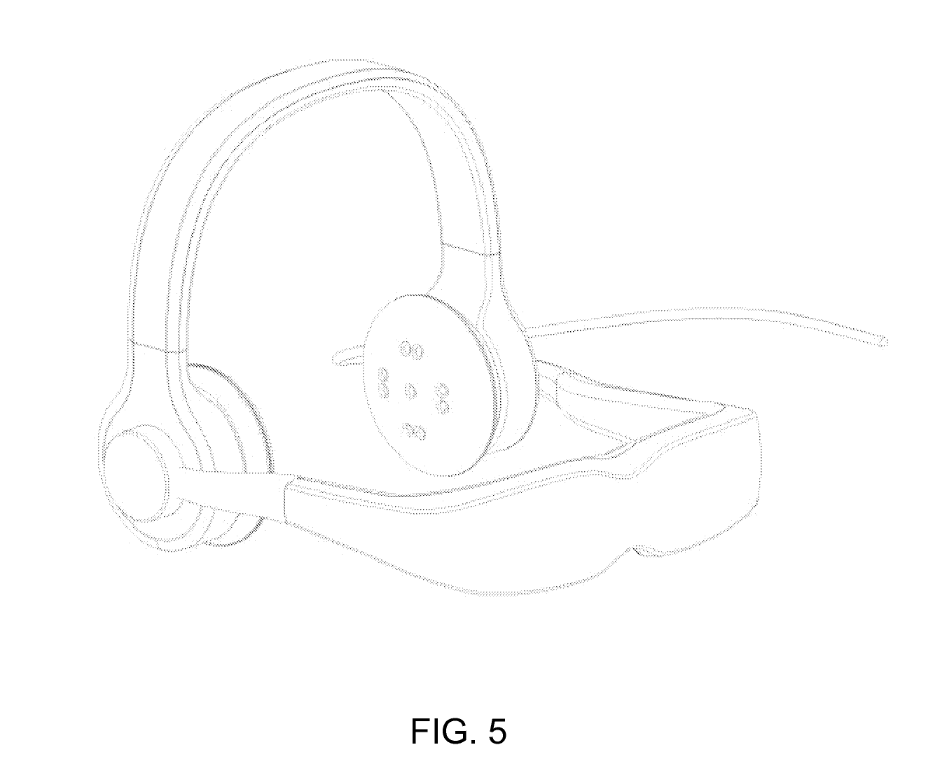

FIG. 6 shows bottom and top views of the device of FIG. 1.

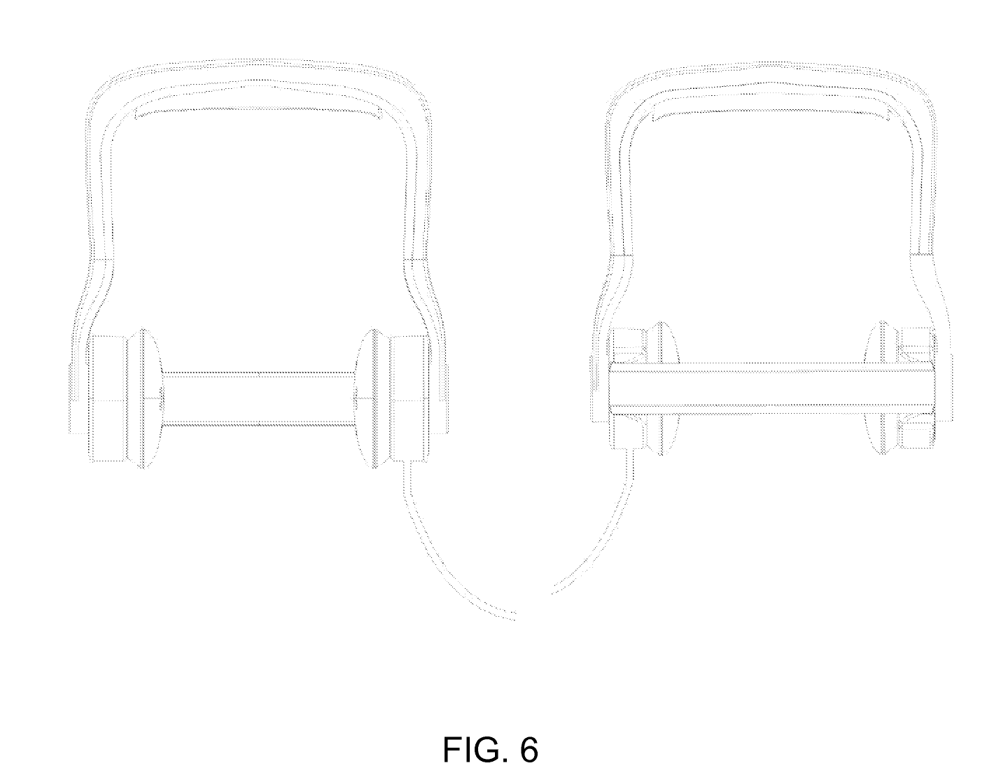

FIG. 7 another perspective view of the device of FIG. 1.

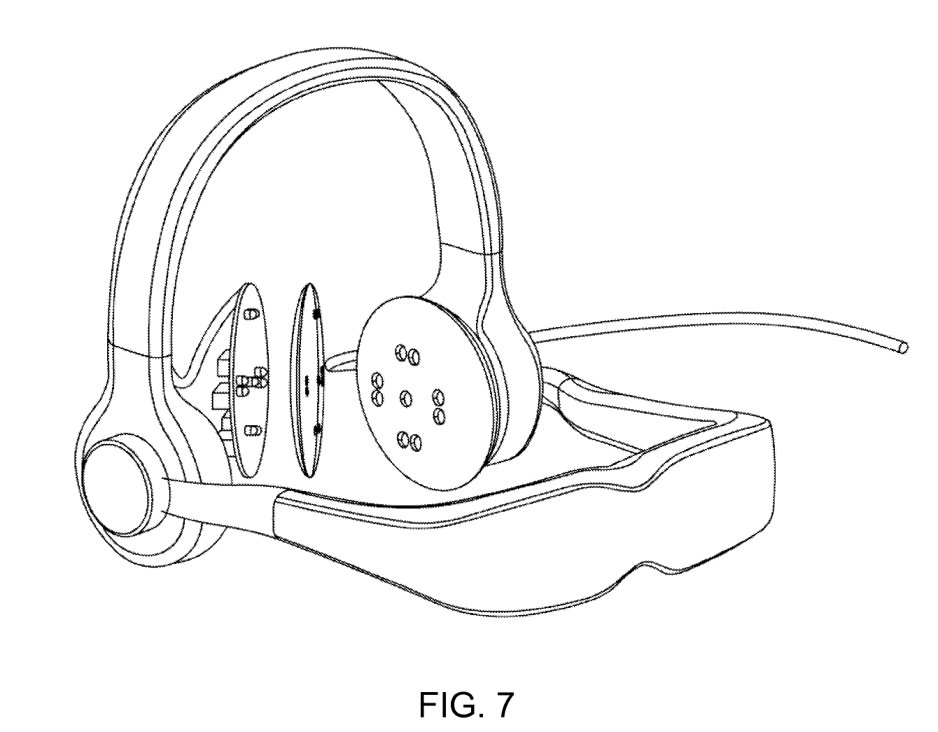

FIG. 8 shows a view of the device of FIG. 1 with control unit.

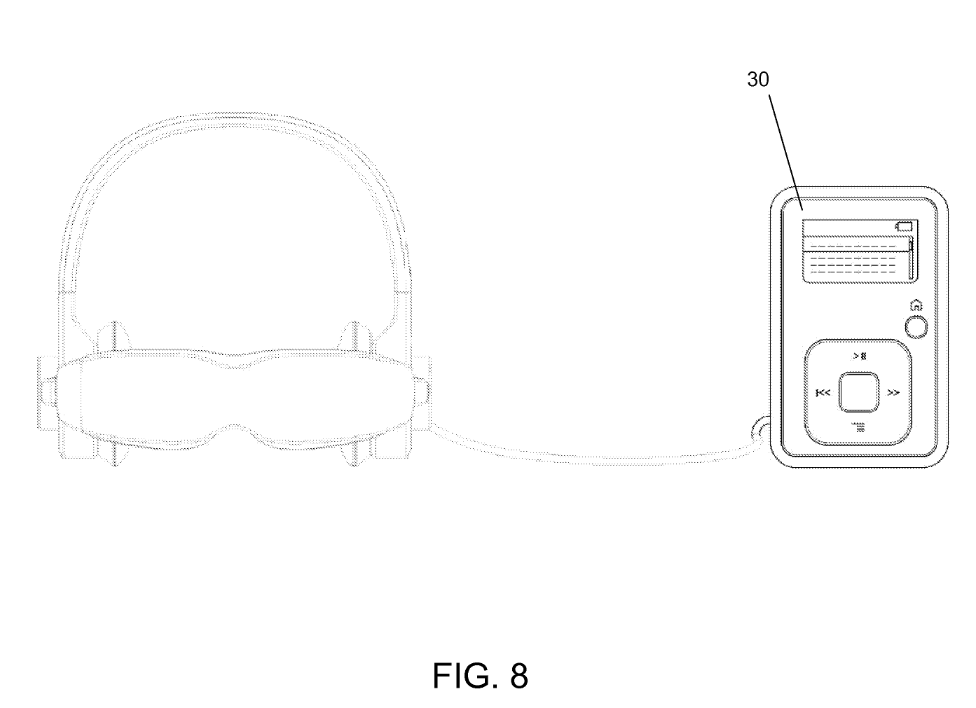

### DETAILED DESCRIPTION OF EXEMPLARY EMBODIMENTS

In various embodiments, the present invention comprises an eyeglass device (which can be in the form of an eye mask, pair of eyeglasses, a pair of goggles, or a form of face plate) with a plurality of semiconductor light and/or vibrational sources (collectively, “light sources”), such as, but not limited to, light-emitting diodes (LEDs) or laser diodes, fiber optics, full spectrum light sources, piezo transducers, vibrational transducers, or a combination thereof, that provide therapeutic and rejuvenating effects to human tissue. The light sources emit energy in the form of photons when switched on, which delivers energy to targeted tissue, penetrating the layers of skin to produce a non-thermal photochemical effect at the cellular level. The therapy is noninvasive, and avoids the potential side effects of older forms of therapy, such as drug therapy.

In one embodiment, the present invention comprises a phototherapy eyeglass device 2 with a plurality of light sources 16 arrayed on an interior-facing face plate 10 via magnets, VHB tape, and/or any other means. The face plate may be detachable. Various face plates contain various configurations or patterns of light sources, in a variety of colors and intensities (i.e., milliwatt range). Examples of light source orientation thus include, but are not limited to, a fixed straight position or a variety of angled positions (i.e., with respect to the face plate) allowing bi-directional ability of light to crisscross. The face plates also may vary in size and shape. Different colors may be used for different treatments. The face plates are interchangeable by the user as desired.

The eyeglass device can be in a variety of forms, including a simple eyeglass or goggle configuration with arms to fit behind the ears, or a strap that fits around the head. In an alternative configuration, the device may be secured by a strap or band that fits over the top of the head, as seen in the figures. In this latter configuration, one or two ear buds or “earphones” 20 may be attached in the appropriate position, as shown. The ear buds or earphones may comprise speakers to provide music, radio, or other sound while the user is wearing the device.

In one exemplary embodiment, the ear buds or earphones further comprise light sources 16 (as described above). The device also may contain optical fibers to carry light to the ear buds or earphones. Light sources may be placed in a variety of configurations or patterns, in a similar manner as described above, to allow light to enter through the ears and thereby impact the inner ear and brain, or to illuminate the area around and outside of the ear. Removable faceplates, as described above, may also be used with the earbuds or earphones.

The device can comprise a number of individual light sources. The configuration or patterns of light sources can vary, as well as the color and intensities (i.e., milliwatt ranges) of the light sources. Likewise, the light sources can comprise various mixes of types of light sources (e.g., all LEDs, all laser diodes, all fiber optics, or a combination thereof). Different colors and intensities may be used for different treatments. In addition, different colors and intensities may be used in different areas.

The light sources can be controlled by wireless or wired connection with a computing device operating a control program, a touchpad monitor or the like on or connected to the eyeglass device, or a control unit 30. The user can control color, type, duration, wavelength amplitude, wavelength phase, and frequency (pulse) of the light sources being activated during therapeutic application, as described in further detail below. For portable operation, light and frequency settings as well as visualization recordings may be embedded into the unit itself through a scan disk or can be uploaded from a software device to contain specific frequencies and light patterns. The phototherapy eyeglass device has the capacity to accept a scan disk drive or similar technology to store information such as music, MP3 files, and any other type of material for use with the device. In addition, such media and encodings may be transferred from one device to another.

Power for the invention may be provided by plugging the device into a standard electrical outlet. Alternatively, one or more batteries may be provided.

In one embodiment, the present invention utilizes RBG technology. RBG technology utilizes the color mixing properties of red, green, and blue LED chips that are provided on a reflector. A photomixing material and filler resin scatters the light rays to uniformly combine the rays emitted from the LED chips. The photomixing material and filler resin are applied onto upper sides of the light emitting diode chips while being mixed with each other, and the photomixing material is uniformly dispersed in the filler resin.

In several embodiments, the light sources include red, blue, green and orange colors. These colors have the following effects:
Red: stimulates vitality and growth; good for fatigue and debilitating conditions; use for deficient nutrition, dormant conditions, poor appetite, constipation, depression, drowsiness, and paralysis.
Blue: slows down growth; calming; acts as a sedative; relieves excitement and inflammation; resets “biological clock” of the human body using doses of 20 minutes; blue light to Alzheimer's patients helped biological clock to sleep longer at night; use for nervousness, irritability, fussiness, feverishness; apply to all conditions where inflammation is present; use for internal bleeding, nervous conditions.
Green: slows down growth; calming; relieves excitement and inflammation; useful when combined with blue/red and yellow as a brain/nerve stimulate and laxative.
Orange: a combination of red and yellow is powerful in colds and sluggish/chronic conditions as it helps release stored energy.

In one embodiment, the invention can run any light frequency within the pulsing range of 0 Hz to 100,000,000,000 Hz, and can run all safe wavelengths of the electromagnetic spectrum, including visible light and near-infrared light. In one exemplary embodiment, the power output per laser diode, LED, or fiber optic ranges from 1 mW to 300 mW.

In another exemplary embodiment, the invention produces specific wavelengths in the form of linear waves, including, but not limited to, sine, square, triangular, and saw tooth waves. Additional wave types can be produced, including, but not limited to, solution waves, a self-reinforcing solitary wave (a wave packet or pulse) that maintains its shape while it travels at constant speed, and longitudinal waves capable of passing through tissue from one side to another with no loss of strength. The utilization of a single wave type or combination of wave types produces a wavelength with no degradation to wave shape allowing the energy produced from the wave to penetrate any desired depth of biological tissue.

Using the applicable control mechanism, such as the control box 30, in several embodiments the operator or user can control the frequency, amplitude, and phase of the wavelengths through a digital interface. The operator can select the frequency (pulse), wavelength, amplitude, and wave type associated with each light-emitting source. This phase relationship allows for each channel to be specifically programmed with frequency and peak-to-peak amplitude allowing multiple channels to operate at a different frequency (pulse) and amplitude. Thus, the phototherapy device can produce multiple wavelengths, multiple wave types, multiple frequencies (pulses), and multiple amplitudes.

In one embodiment, the control box comprises a front and back, which may be glued, affixed or screwed together. The interior comprises the control electronics and light power supply, a wireless chip, a “handshake” chip, a MP3 player, and a RF or Bluetooth smart chip for identification of the unit. The control box may comprise a power indicator, a power switch, an USB port (for updating software) and other controls, switches and ports as necessary. The control box may be made in a variety of colors, sizes, and configurations.

In a further embodiment, the present invention incorporates the geometric configuration of single light technology or multiple light technologies, wavelength, amplitude and power output, referred to as the array. The geometric configuration is not limited to any single configuration and can include any geometric configuration of wavelengths, power output, amplitude, and wave types. The array is engineered to produce multiple wavelengths, power outputs, amplitudes and wave types producing therapeutic benefits to biological tissue. This is accomplished by utilizing expandable software, smart chips, adaptive lenses, and light producing technology that is completely scalable and configurable to operator needs. The geometric arrangement of the light technology is not limited to any single geometric configuration, wavelength, power output, amplitude, or wave type. The array can be configured to support any geometric configuration of multiple wavelengths, multiple power outputs, multiple amplitudes, or multiple wave types.

In one particular embodiment, the present invention will only work with specific control boxes, accessories, or computing devices, which can be self-identifying through “handshake” communications technology. Utilizing handshake technology will only pair specific units to specific accessories. A specific circuit board chip may be utilized in each and every piece of equipment. These chips include a one-of-a-kind code that forms a unique link to each other. The circuit board may be a variable frequency circuit board. Specific RF chips may be installed in each and every unit so that identifications can be placed into each piece of equipment to identify purchase dates and other necessary information.

In yet a further embodiment, the invention is equipped with an USB port and wireless circuit board that will operate and control peripheral devices by the digital interface of the phototherapy device. Peripheral devices are not limited to and include light technology devices and any device that generates frequency or electrical pulse. The peripheral devices will be activated upon a passcode entered into the digital interface of the phototherapy device. In addition, an internal solid-state hard drive may be included in each unit to provide storage for music and other audio files.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A phototherapy device for light therapy applications, comprising:
a face plate with an interior and an exterior;
a plurality of light sources located on the interior of the face plate.

2. The device of claim 1, further comprising a strap adapted to hold the face plate in place.

3. The device of claim 1, further comprising an eyeglass frame to which the face plate is removably attached.

4. The device of claim 1, further comprising a goggles frame to which the face plate is removably attached.

5. The device of claim 1, further comprising a control unit adapted to control the plurality of light sources.

6. The device of claim 1, further comprising one or more vibrational sources located on the face plate.

7. The device of claim 1, further comprising one or more ear buds or earphones.

8. The device of claim 7, further comprising one or more light or vibrational sources, or combinations thereof, located on or in said ear buds or earphones.

9. A method of providing light therapy to the eyes or face, comprising:
placing a phototherapy device on the face or portion thereof, said phototherapy device comprising a face plate with an interior and an exterior, with a plurality of light sources, alone or in combination with one or more vibrational sources, located on the interior of the face plate; and
applying various frequencies and intensities or light from said light sources to the portion of the face covered by the face plate.

10. An apparatus, comprising:
an pair of eyeglasses or goggles with an area adapted to cover the eyes of a user; and
a plurality of light sources located on an interior of the area adapted to cover the eyes of the user.

11. The apparatus of claim 10, further comprising one or more ear buds or earphones affixed to said pair of eyeglasses or goggles.

12. The apparatus of claim 10, further comprising one or more vibrational sources affixed to said pair of eyeglasses or goggles.

13. The apparatus of claim 10, further comprising a control unit adapted to control the plurality of light sources.
